(19) United States
(12) Patent Application Publication
(10) Pub. No.: US 2026/0112506 A1
(43) Pub. Date: Apr. 23, 2026

(54) SYSTEMS AND METHODS FOR PROCESSING PROFILES INCLUDED IN A PROTECTED DATASET MAINTAINED IN A SECURED NETWORK LOCATION TO DETERMINE CORRELATIONS BETWEEN INDICATORS REPRESENTING LATENT PATTERNS THAT ARE INDICATIVE OF A CONDITION

(71) Applicant: AML JV, LLC, Aurora, CO (US)

(72) Inventors: Justin DALE, Aurora, CO (US); Frank MARKSON, Aurora, CO (US); Brett STEVENS, Aurora, CO (US); Austin GILLEN, Aurora, CO (US); Md Nazmul ISLAM, Aurora, CO (US)

(73) Assignee: AML JV, LLC, Aurora, CO (US)

(21) Appl. No.: 19/364,650

(22) Filed: Oct. 21, 2025

Related U.S. Application Data
(60) Provisional application No. 63/710,557, filed on Oct. 22, 2024.

(51) Int. Cl. G16H 50/70

(52) U.S. Cl. CPC G16H 50/70

(57) ABSTRACT

Described herein are systems and methods for determining correlations between indicators. In an example, a system can be configured to obtain data associated with a set of individual profiles corresponding to a set of individuals and identify individual profiles indicative of a condition based on a first biomarker indicative of the condition being satisfied by the individual profiles. The system can then determine a set of attributes among each of the individual profiles and determine correlations between subsets of the attributes. In examples, the correlations can be indicative of a subset of biomarkers different from the first biomarker that are indicative of the condition.

### CROSS-REFERENCE TO RELATED APPLICATION

The present application claims the benefit of, and priority to, U.S. Provisional Patent Application No. 63/710,557 , filed Oct. 22, 2024, the entire contents of which are hereby incorporated by reference in their entirety for all purposes.

### TECHNICAL FIELD

This application generally relates to techniques for processing profiles included in a protected dataset maintained in a secured network location to determine correlations between indicators representing latent patterns that are indicative of a condition and, in some embodiments, to techniques for analyzing indicators (e.g., biomarkers) across individual profiles (e.g., treatment profiles) to determine correlations between biomarkers representing latent patterns indicative of a condition.

### BACKGROUND

Currently, conditions (e.g., diseases) such as acute myeloid leukemia (AML) are treated in coordination with treatment plans developed by clinicians and based on standard of care therapies. These treatment plans can include the use of targeted therapies such as administration of drugs targeting specific molecules or pathways involved in the growth and survival of the disease, stem cell transplants, and/or the like. Typically, these treatments are administered in conjunction with monitoring of the response of the patient through diagnostic testing and updated to optimize a patient's outcome. But it is often difficult for clinicians to identify AML at earlier stages as the symptoms (e.g., fatigue, fever, weight loss) can be attributed to many common and less severe conditions. This can lead to therapies being applied to patients later in the progression of the disease that are less efficient and that do not result in an optimal outcome.

### SUMMARY

Embodiments described herein include systems and methods for processing profiles included in a protected dataset maintained in a secured network location to determine correlations between indicators representing latent patterns that are indicative of a disease condition being present in patients and can provide any number of additional or alternative benefits as well.

In some embodiments, a system for processing profiles included in a protected dataset maintained in a secured network location and configured to prevent re-identification of individuals represented by the profiles to determine correlations between indicators representing latent patterns that are indicative of a condition being present in individuals may comprise one or more processors configured to: obtain data associated with a set of individual profiles corresponding to a set of individuals, where each individual profile of the set of individual profiles comprises a plurality of entries representing a state of an individual; determine a set of attributes associated with each individual corresponding to each individual profile, where each attribute is associated with at least one indicator representing the state of the individual; in response to determining that a first indicator is present for the set of individuals based on the first indicator satisfying an expression level for each individual, determine correlations between a subset of the attributes for each individual of the set of individuals that are being treated for a condition, the correlations indicating a subset of indicators different from the first indicator that are indicative of the condition; and provide output data associated with the correlations indicating the subset of indicators, the output data configured to cause a graphical user interface (GUI) to be displayed that indicates subset of indicators that are indicative of the condition.

The one or more processors configured to determine the correlations between the subset of the attributes may be configured to: cluster the individual profiles to form a plurality of clusters, where each individual profile is assigned to a cluster of the plurality of clusters based on similarities between attributes of the subset of attributes, and determine the correlations between the subset of the attributes based on the plurality of clusters.

The one or more processors configured to determine the correlations between the subset of the attributes based on the plurality of clusters may be configured to: perform a cluster analysis based on the plurality of clusters to identify attributes that are indicative of presence of the condition, and determine the subset of indicators that correspond to the attributes that are indicative of the presence of the condition.

The one or more processors configured to determine the correlations between the subset of the attributes may be configured to: determine a first set of correlations indicating a first subset of indicators, where each indicator of the first subset of indicators satisfies a primary correlation threshold indicative of the condition, and wherein the one or more processors are further configured to: determine a second set of correlations indicating a second subset of indicators, where each indicator of the second subset of indicators satisfies a secondary correlation threshold indicative of the condition, and where each indicator of the second subset of indicators is not indicated by the first subset of indicators.

The one or more processors configured to obtain the data associated with a set of individual profiles corresponding to a set of individuals may be configured to: obtain the data associated with a set of individual profiles, where each individual profile comprises a plurality of entries having one or more sub-entries that represent the state of the individual over a period of time, and wherein the one or more processors configured to determine the correlations between the subset of the attributes are configured to: determine correlations between the subset of the attributes for individuals over the period of time, the correlations indicating changes in the subset of indicators that are indicative of the condition.

The one or more processors configured to determine the correlations between the subset of the attributes for individuals over the period of time may be configured to: determine a first attribute that is associated with a change pattern over the period of time, the change pattern representing changes in quantity of an indicator represented by samples gathered from individuals over the period of time.

The one or more processors configured to determine the correlations between the subset of the attributes for individuals over the period of time may be configured to: determine a first attribute that is associated with a first change pattern over the period of time, the first change pattern representing first changes in quantity of a first indicator represented by samples gathered from individuals over the period of time; determine a second attribute that is associated with a second change pattern over the period of time, the second change pattern representing first changes in quantity of a second indicator represented by samples gathered from individuals over the period of time; and determine a correlation between the first attribute and the second attribute based on a comparison of the first changes in quantity and the second changes in quantity over time.

The first attribute may be further associated with a correlation that satisfies a primary correlation threshold indicative of the condition, and the second attribute may be further associated with correlation that satisfies the primary correlation threshold or a secondary correlation threshold indicative of the condition.

In some embodiments, a method for processing profiles included in a protected dataset maintained in a secured network location and configured to prevent re-identification of individuals represented by the profiles to determine correlations between indicators representing latent patterns that are indicative of a condition being present in individuals may comprise: obtaining, by one or more processors, data associated with a set of individual profiles corresponding to a set of individuals, where each individual profile of the set of individual profiles comprises a plurality of entries representing a state of an individual; determining, by the one or more processors, a set of attributes associated with each individual corresponding to each individual profile, where each attribute is associated with at least one indicator representing the state of the individual; in response to determining that a first indicator is present for the set of individuals based on the first indicator satisfying an expression level for each individual, determining, by the one or more processors, correlations between a subset of the attributes for each individual of the set of individuals that are being treated for a condition, the correlations indicating a subset of indicators different from the first indicator that are indicative of the condition; and providing, by the one or more processors, output data associated with the correlations indicating the subset of indicators, the output data configured to cause a graphical user interface (GUI) to be displayed that indicates subset of indicators that are indicative of the condition.

Determining the correlations between the subset of the attributes may comprise clustering, by the one or more processors, the individual profiles to form a plurality of clusters, where each individual profile is assigned to a cluster of the plurality of clusters based on similarities between attributes of the subset of attributes, and determining, by the one or more processors, the correlations between the subset of the attributes based on the plurality of clusters.

Determining the correlations between the subset of the attributes based on the plurality of clusters may comprise performing, by the one or more processors, a cluster analysis based on the plurality of clusters to identify attributes that are indicative of presence of the condition, and determine, by the one or more processors, the subset of indicators that correspond to the attributes that are indicative of the presence of the condition.

Determining the correlations between the subset of the attributes may include determining, by the one or more processors, a first set of correlations indicating a first subset of indicators, where each indicator of the first subset of indicators satisfies a primary correlation threshold indicative of the condition, and the method may further comprise determining, by the one or more processors, a second set of correlations indicating a second subset of indicators, where each indicator of the second subset of indicators satisfies a secondary correlation threshold indicative of the condition, and where each indicator of the second subset of indicators is not indicated by the first subset of indicators.

Obtaining the data associated with a set of individual profiles corresponding to a set of individuals may comprise obtaining, by the one or more processors, the data associated with a set of individual profiles, where each individual profile comprises a plurality of entries having one or more sub-entries that represent the state of the individual over a period of time, and wherein determining the correlations between the subset of the attributes may comprise determining, by the one or more processors, correlations between the subset of the attributes for individuals over the period of time, the correlations indicating changes in the subset of indicators that are indicative of the condition.

Determining the correlations between the subset of the attributes for individuals over the period of time may comprise determining, by the one or more processors, a first attribute that is associated with a change pattern over the period of time, the change pattern representing changes in quantity of an indicator represented by samples gathered from individuals over the period of time.

Determining the correlations between the subset of the attributes for individuals over the period of time may comprise determining, by the one or more processors, a first attribute that is associated with a first change pattern over the period of time, the first change pattern representing first changes in quantity of a first indicator represented by samples gathered from individuals over the period of time; determining, by the one or more processors, a second attribute that is associated with a second change pattern over the period of time, the second change pattern representing first changes in quantity of a second indicator represented by samples gathered from individuals over the period of time; and determining, by the one or more processors, a correlation between the first attribute and the second attribute based on a comparison of the first changes in quantity and the second changes in quantity over time.

The first attribute may be further associated with a correlation that satisfies a primary correlation threshold indicative of the condition, and wherein the second attribute may be further associated with correlation that satisfies the primary correlation threshold or a secondary correlation threshold indicative of the condition.

In some embodiments, a non-transitory computer-readable medium storing instructions thereon that, when executed by one or more processors, may cause the one or more processors to: obtain data associated with a set of individual profiles corresponding to a set of individuals, where each individual profile of the set of individual profiles comprises a plurality of entries representing a state of an individual; determine a set of attributes associated with each individual corresponding to each individual profile, where each attribute is associated with at least one indicator representing the state of the individual; in response to determining that a first indicator is present for the set of individuals based on the first indicator satisfying an expression level for each individual, determine correlations between a subset of the attributes for each individual of the set of individuals that are being treated for a condition, the correlations indicating a subset of indicators different from the first indicator that are indicative of the condition; and provide output data associated with the correlations indicating the subset of indicators, the output data configured to cause a graphical user interface (GUI) to be displayed that indicates subset of indicators that are indicative of the condition.

The instructions that cause the one or more processors configured to determine the correlations between the subset of the attributes may cause the one or more processors to: cluster the individual profiles to form a plurality of clusters, where each individual profile is assigned to a cluster of the plurality of clusters based on similarities between attributes of the subset of attributes, and determine the correlations between the subset of the attributes based on the plurality of clusters.

The instructions that cause the one or more processors configured to determine the correlations between the subset of the attributes based on the plurality of clusters may cause the one or more processors to: perform a cluster analysis based on the plurality of clusters to identify attributes that are indicative of presence of the condition, and determine the subset of indicators that correspond to the attributes that are indicative of the presence of the condition.

The instructions that cause the one or more processors configured to determine the correlations between the subset of the attributes may cause the one or more processors to: determine a first set of correlations indicating a first subset of indicators, where each indicator of the first subset of indicators satisfies a primary correlation threshold indicative of the condition, and wherein the instructions further cause the one or more processors to: determine a second set of correlations indicating a second subset of indicators, where each indicator of the second subset of indicators satisfies a secondary correlation threshold indicative of the condition, and where each indicator of the second subset of indicators is not indicated by the first subset of indicators.

When implemented, these systems and methods can address inefficiencies involved in conventional approaches to diagnosing conditions (e.g., diseases), allowing for faster and more accurate diagnosis and treatment. More specifically, techniques implemented based on the systems and methods described herein can allow for the correlation of attributes known to be indicative of a given condition (e.g., specific indicators associated with biomarkers indicative of the condition) with other attributes or combinations of attributes that are not known to be indicative of the condition (e.g., biomarkers not known to be indicative of the condition). For example, where a condition is associated with expression of a certain protein, individuals may be observed as presenting symptoms that indicate any number of possible issues, from less critical issues (e.g., fatigue) to more critical issues (e.g., the presence of conditions such as cancer). The present disclosure improves on conventional approaches by providing a framework for analyzing and drawing correlations between indicators known to be indicative of a condition with other indicators or combinations of indicators that can, for example, be obtained through routine individual testing. As a result, individuals can be screened more quickly for these conditions using, in some cases, more common or routine diagnostic tests and, when diagnosed, be offered treatment faster than would otherwise be the case. And, by extension, individual health outcomes can be improved (e.g., through extension of life).

Further, because many early symptoms of conditions (e.g., diseases) can be difficult to identify or attributed to a wide variety of other common issues (e.g., stress, common illnesses, and/or the like), the systems and methods described herein can be implemented to identify latent (e.g., imperceptible) patterns across indicators that would not otherwise be identifiable by clinicians. Once these patterns (or correlations) are identified, systems involved in diagnosing individuals can be configured to diagnose subsequent individuals with the condition when the latent patterns are present in subsequently-received individual profiles of the individuals. This, again, can result in the offering of treatment faster than would otherwise be possible and similar chances of improving patient health outcomes through earlier diagnosis.

It is to be understood that both the foregoing general description and the following detailed description are exemplary and explanatory, and are intended to provide further explanation of the embodiments described herein.

### BRIEF DESCRIPTION OF THE DRAWINGS

The accompanying drawings constitute a part of this specification, illustrate one or more embodiments and, together with the specification, explain the subject matter of the disclosure.

FIG. 1 illustrates a block diagram of an environment, in accordance with one or more embodiments described herein.

FIG. 2 illustrates a flow diagram illustrating operations of a method for analyzing biomarkers to determine interdependencies indicative of a condition, in accordance with one or more embodiments described herein.

FIG. 3A-3D illustrate a diagram of an example implementation of the method of FIG. 2, in accordance with one or more embodiments described herein.

### DETAILED DESCRIPTION

Reference will now be made to the embodiments illustrated in the drawings, and specific language will be used here to describe the same. It will nevertheless be understood that no limitation of the scope of the disclosure is thereby intended. Alterations and further modifications of the features illustrated here, and additional applications of the principles as illustrated here, which would occur to a person skilled in the relevant art and having possession of this disclosure, are to be considered within the scope of the disclosure.

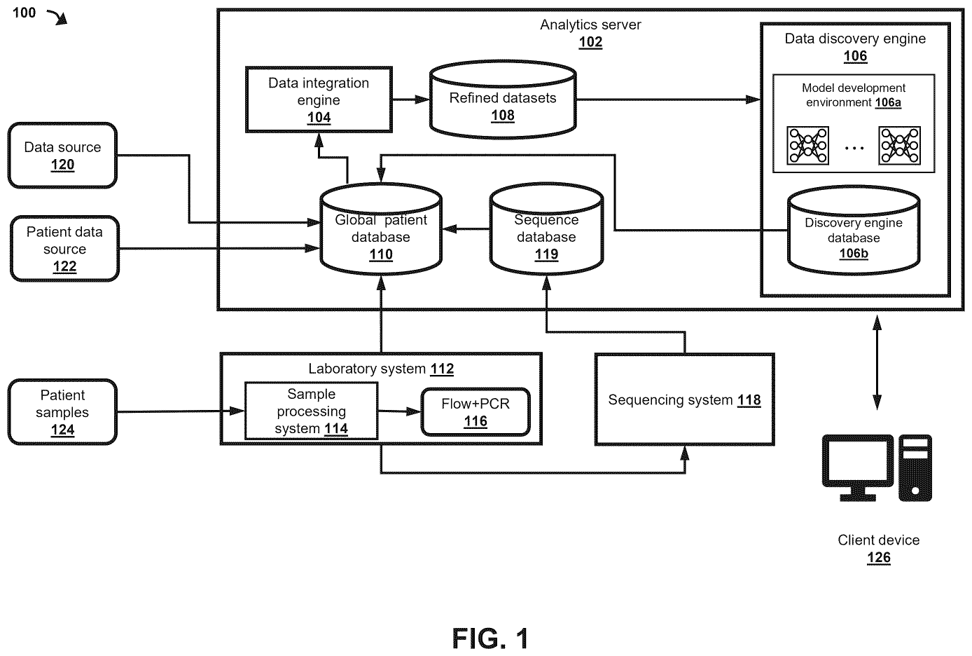

FIG. 1 is a block diagram of an environment 100 for managing patient data, according to an embodiment. The environment 100 can include an analytics server 102, a laboratory system 112, a sequencing system 118, a data source 120, patient data source 122, patient samples 124, and a client device 126. Various components depicted in FIG. 1 can belong to an organization involved in clinical research of one or more conditions such as, for example, acute myeloid leukemia (AML) or other conditions and/or to one or more organizations involved in treating patients with the one or more conditions. While certain components and devices are illustrated as being included in the environment 100 of FIG. 1, it will be understood that the environment 100 is not confined to the components or conditions as described herein and can include additional or different components (not shown for purposes of brevity and clarity) which are configured to be considered within the scope of the embodiments described herein.

In some embodiments, the analytics server 102 can include any computing device comprising a processor and non-transitory machine-readable storage capable of executing the various tasks, processes, and/or operations as described herein. The analytics server 102 can employ various processors such as central processing units (CPUs), graphical processing units (GPUs), and/or the like. Some non-limiting examples of such computing devices can include workstation computers, laptop computers, server computers, and/or the like. While the environment 100 includes a single analytics server 102, there can be multiple analytics servers 102. Further, the analytics server 102 can include any number of computing devices operating in a distributed computing environment such as, for example, a cloud computing environment. As described herein, the analytics server 102 can include a data integration engine 104, a data discovery engine 106, refined datasets 108, a global patient database 110, and a sequence database 119. In some embodiments, the analytics server 102 can include and/or implement operations that are associated with the laboratory system 112, the sequencing system 118, and/or the client device 126. In some embodiments, the analytics server 102 can include and/or implement operations that are associated with (e.g., involved in the generation of) the data source 120, the patient data source 122, and/or the patient samples 124.

In some embodiments, the analytics server 102 can be configured to receive data from the data source 120, the patient data source 122, the laboratory system 112, and sequencing system 118 when processing patient samples 124. For example, the analytics server 102 can be configured to receive data from the data source 120, where the data is associated with (e.g., represents) entries corresponding to one or more patient files. As an example, as patients interact with clinicians, the clinicians can generate information that are received as input at a client device (not explicitly illustrated) that is associated with the clinicians, the notes indicating clinical observations and/or updates to treatment plans for the patients made by the clinician. The client device can then generate patient data that is associated with each patient and representative of the clinical observations or updates to the treatment plans and store the patient data in the data source 120 to later transmit to the analytics server 102. In this example, the analytics server 102 can implement the global patient database 110 such that the patient data is uploaded and stored in the global patient database 110 in association with one or more identifiers for the patient as described herein.

In another example, the analytics server 102 can be configured to receive data from the patient data source 122, where the data is associated with (e.g., represents) information about individual patients. As an example, as a history of a patient is obtained, the clinicians and/or the patients can generate information that is received as input at a client device (not explicitly illustrated) that is associated with the clinicians and/or patients, the information indicating aspects of the history of the patient such as whether the patient is associated with a history of a given disease in their family, whether the patient had any exposure to environmental conditions associated with the given disease, and/or the like. The client device can then generate patient data that is associated with each patient and representative of the history of the patient and store the patient data in the patient data source 122 to later transmit to the analytics server 102. In this example, the analytics server 102 can obtain and store the patient data in the global patient database 110 in association with one or more identifiers for the patient as described herein.

In yet another example, the analytics server 102 can be configured to receive data from the laboratory system 112 and/or the sequencing system 118, where the data is associated with (e.g., represents) information about patient samples (e.g., tissue samples, blood samples, blood counts (e.g., complete blood counts), bone marrow aspiration and biopsy results, lumbar puncture results, and/or the like) as well as the results of the processing of the samples (e.g., a DNA sequence or targets thereof). As an example, as a patient is evaluated and/or treated for a disease such as AML, patient samples 124 similar to those described above can be obtained. The patient samples 124 can be initially obtained and processed by a laboratory system 112 and processed by a sample processing system 114. The sample processing system 114 can implement one or more devices configured to obtain and store the patient samples and extract DNA from the patient samples. For example, in preparation for genetic analysis to guide AML treatment, patient blood or bone marrow can first be obtained from a patient and frozen. Later, these samples can be quality checked to ensure the sample purity and quantity are sufficient for sequencing. In some embodiments, the isolated DNA can then undergo further processing to be separated into manageable fragments and equipped with adapters (e.g., short, specific pieces of synthetic DNA associated with the fragmented DNA molecules) for compatibility with sequencing machines. In some embodiments, the samples can also be provided to a flow and polymerase chain reaction (PCR) system to extract and amplify the isolated DNA. The laboratory system 112 can then provide the processed samples and corresponding data representing the samples to be processed by the sequencing system 118. Additionally, or alternatively, the laboratory system 112 can then provide the data generated by the laboratory system 112 when processing the samples to the analytics server 102 to be stored in the global patient database 110.

In some embodiments, the sequencing system 118 can be configured to receive the patient samples and/or the isolated DNA and sequence the patient samples. In one example, the sequencing system 118 can attach DNA fragments to a surface in a specific pattern, creating clusters. The sequencing itself can involve a series of cycles where fluorescently labeled nucleotides are introduced one by one. The incorporation of each base can be detected, identifying the sequence of the fragment base by base. Finally, the sequencing system 118 can analyze the vast amount of data, assemble the original DNA sequences and identify any variations or mutations present (sometimes referred to as Next-Generation Sequencing (NGS)). The sequencing system 118 can then provide data associated with the sequenced DNA to the analytics server 102. In this example, the analytics server 102 can store the sequenced DNA in a sequence database 119 that stores the sequenced DNA in association with one or more patient identifiers established by the analytics server 102. In some embodiments, the analytics server 102 can also cause the sequence database 119 to provide the data associated with the sequenced DNA to the global patient database 110 to be stored in association with other data associated with the patient such as a treatment profile and/or limited treatment profile (referred to as an individual profile and/or a limited individual profile) for the patient as described herein.

In some embodiments, the analytics server 102 can implement a data integration engine 104 to process data stored in the global patient database 110. For example, the analytics server 102 can implement the data integration engine 104 such that the data integration engine 104 is configured to obtain the data associated with the patients that is stored in the global patient database 110 and processes the data to be used by the data discovery engine 106. In one example, as data is obtained by the global patient database 110 for a given patient, the data can be stored in the global patient database 110 in association with one or more identifiers as part of a profile for the patient. The data integration engine 104 can then obtain the data associated with the patient (e.g., the entire profile or portions thereof) from the global patient database 110 and process the data to generate a limited treatment profile. The limited treatment profile can then be stored in the refined datasets 108 (referred to herein as “refined datasets”) and made available to the data discovery engine 106. In this way, the analytics server 102 can maintain two separate datasets that allow for updates to the limited treatment profiles stored in the refined datasets 108 and subsequent use by the data discovery engine 106 when performing the operations described herein. As will be understood, in this example the data associated with the patient that is stored in the global patient database 110 can be updated over time such that the patient profile is represented as a set of entries associated with a time series. As the global patient database 110 is updated, the data integration engine 104 can obtain updated versions of the data associated with the patient from the global patient database 110, process the data when updating the limited treatment profiles in the refined datasets 108, and store the updates in the refined datasets 108.

In some embodiments, the analytics server 102 can implement the data discovery engine 106 that includes a model development environment 106a and a discovery engine database 106b. For example, the analytics server 102 can implement the data discovery engine 106 such that the data discovery engine 106 is configured to receive data associated with one or more limited treatment profiles that are stored in the refined datasets 108 (which can include a protected dataset that is maintained in a secured network location associated with (e.g., established by) the analytics server 102) and process the one or more limited treatment profiles. In this example, the analytics server 102 can process the one or more limited treatment profiles using the model development environment 106a. Processing the limited treatment profiles can include providing the limited treatment profiles to one or more models (e.g., machine learning-based models and/or the like) to determine one or more metrics corresponding to the performance of each of the models to indicate which model is most accurate, efficient, and/or the like at generating one or more predictions. These predictions can include indications of treatment options that have a likelihood of optimizing an outcome (e.g., lifespan) for the patients. Processing the limited treatment profiles can additionally, or alternatively, include determining one or more aspects of the limited treatment profiles. For example, where the limited treatment profiles is associated with a predetermined number of possible attributes but the patient samples 124 obtained to be processed were limited and only usable to determine a subset of the possible attributes, the model development environment 106a can process the portions of the refined patient profile that are available in the refined datasets 108 to determine one or more of the remaining attributes. In this example, data associated with the one or more remaining attributes can be stored by the data discovery engine 106 in the discovery engine database 106b. The analytics server 102 can then periodically or in real-time update the global patient database 110 based on the data associated with the limited treatment profiles (e.g., the one or more remaining attributes and/or the like) that are stored in the discovery engine database 106b.

In some embodiments, the client device 126 can include any computing device comprising a processor and non-transitory machine-readable storage capable of executing the various tasks, processes, and/or operations as described herein. The client device 126 can employ various processors such as central processing units (CPUs), graphical processing units (GPUs), and/or the like. Some non-limiting examples of such computing devices can include workstation computers, laptop computers, server computers, and/or the like. While the environment 100 includes a single client device 126, there can be multiple client devices 126. Further, the client device 126 can include any number of computing devices operating in a distributed computing environment such as, for example, a cloud computing environment. In some embodiments, the client device 126 can be associated with one or more software developers and/or one or more clinicians that are interacting with (e.g., configuring operation of) the analytics server 102 as described herein. In some embodiments, the client device 126 can be associated with one or more clinicians and/or one or more organizations involved in treating patients with the one or more diseases such as a hospital and/or the like.

In some embodiments, the analytics server 102 can generate and display an electronic platform (e.g., via the client device 126) when receiving and processing patient data associated with one or more patients, performing one or more operations when analyzing the patient data, and outputting data associated with the results of the operations performed by any of the components of the analytics server 102 such as, for example, the data discovery engine 106. The electronic platform can include graphical user interfaces (GUI) displayed by display devices of one or more client devices 126. An example of the electronic platform generated and hosted by the analytics server 102 can be a web-based application or a website configured to be displayed on different electronic devices, such as mobile devices, tablets, personal computers, and the like.

In some embodiments, treatment profiles and/or limited treatment profiles may be analyzed to identify trends, commonalities, and divergences across patients or patient subgroups. Such analysis can include direct comparison of temporal treatment sequences, cumulative dosing exposures, treatment intensities, or intervals between successive interventions. By evaluating these patterns, clinicians and researchers may discern which specific treatment pathways or regimen characteristics are consistently associated with improved or diminished outcomes, the analytics server 102 can execute one or more operations to assist with clinical decision-making. In certain cases, composite measures derived from the treatment profiles (e.g., such as dose-density indices, treatment adherence scores, or timing-of-intervention metrics) can be calculated and examined to assess their relationship to patient outcomes. The analysis may additionally include the use of statistical or machine learning algorithms to identify correlations between specific intervention sequences, dosing regimens, or therapeutic combinations and one or more clinical outcome metrics. Such analysis may involve aggregating patient-level treatment history data, mapping these histories against measured outcomes such as overall survival, event-free survival, progression-free survival, or response rates, and applying predictive modeling to determine which profile features are most strongly associated with favorable clinical endpoints. The resulting output generated by the analytics server 102, represented by the treatment profiles, can be used to generate user interfaces that can be displayed (e.g., at the client device 126) to indicate therapies to administer and/or allow for personalized treatment recommendations, optimize protocol design, or adjust ongoing therapy, thereby improving patient prognosis and enhancing resource utilization in clinical practice.

The above-mentioned components can be configured to interconnect with each other and establish communication connections therebetween through a network (not explicitly illustrated). Examples of the network can include, but are not limited to, private or public local-area-networks (LAN), wireless LAN (WLAN) networks, metropolitan area networks (MAN), wide-area networks (WAN), and the Internet. The network can include wired and/or wireless communications according to one or more standards and/or via one or more transport mediums. The communication over the network can be performed in accordance with various communication protocols such as Transmission Control Protocol and Internet Protocol (TCP/IP), User Datagram Protocol (UDP), and IEEE communication protocols. In one example, the network can include wireless communications according to Bluetooth specification sets or another standard or proprietary wireless communication protocol. In another example, the network can also include communications over a cellular network, including, e.g., a GSM (Global System for Mobile Communications), CDMA (Code Division Multiple Access), and EDGE (Enhanced Data for Global Evolution) network.

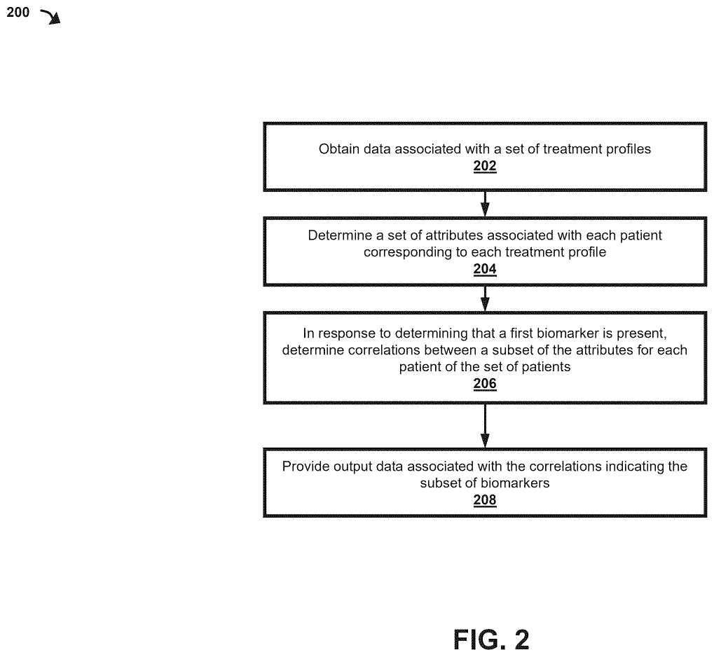

FIG. 2 is a flow diagram illustrating operations of a method 200 for analyzing indicators (e.g., biomarkers) to determine interdependencies indicative of a disease, in accordance with one or more embodiments described herein. In some implementations, one or more of the functions described with respect to method 200 can be performed (e.g., completely, partially, and/or the like) by an analytics server that is the same as, or similar to, the analytics server 102 of FIG. 1. In some implementations, one or more of the functions described with respect to method 200 can be performed (e.g., completely, partially, and/or the like) by another device or group of devices separate from and/or including the analytics server, such as by one or more client devices that are the same as, or similar to, the client device 126 of FIG. 1. For purposes of clarity, the techniques described herein with respect to method 200 are described with respect to analysis of treatment profiles of patients that are the same as, or similar to, the treatment profiles described throughout the application. However, it will be understood that the techniques of method 200 can similarly be applied to limited treatment profiles.

At operation 202, the analytics server can obtain data associated with a set of treatment profiles. For example, the analytics server can obtain data associated with a set of treatment profiles, where each treatment profile corresponds to a patient from among a plurality of patients. The patients can be associated with (e.g., have, be suspected of having, or otherwise be at risk for having) one or more diseases. For example, the patients may have one or more cancers as described herein, for which the patient is being treated. In examples, at least a portion of the patients may not have the one or more diseases. In these examples, the analytics server can determine the treatment profiles that are associated with a given disease or not associated with the disease through comparisons of biomarkers represented by the treatment profiles of the patients and perform one or more of the operations described herein based on the determination that a given patient or set of patients do or do not have the disease.

In some embodiments, each treatment profile can include one or more entries. For example, each treatment profile can include one or more entries that indicate a state of a patient. The state of the patient can represent whether the patient has a disease such as AML, a degree of progression of the disease, and/or the like. In some embodiments, the one or more entries can include values indicating test results generated based on analysis of samples collected from a given patient. The samples can include blood, tissue, and/or the like collected from the patient and processed (e.g., by a laboratory system and/or a sequencing system as described herein). As described herein, the one or more entries can, individually, or in combination, represent a given aspect or set of aspects for the patients. For example, the one or more entries can represent one or more biomarkers that are observed based on the collection and analysis of samples as described herein. In an example, where an aspect is related to an individual biomarker or a set of biomarkers, the combination of biomarkers can represent a state of the patient such as whether the patient has a disease (e.g., AML and/or the like), whether the disease is progressing, and/or the like.

In some embodiments, each entry of a treatment profile can be associated with one or more sub-entries. For example, each entry can be associated with (e.g., include) one or more sub-entries that represent an aspect of the patient over time. In one example, where an entry in a treatment profile includes a biomarker indicating of an expression level of a protein such as CD70, one or more sub-entries can represent relative increases or decreases in the expression of CD70 in the samples of the patient at one or more points in time. The increases or decreases can be represented based on measurements taken at respective points in time over a period of time in which the given patient is monitored. In another example, where a first entry in a treatment profile includes a biomarker indicating an expression level of a protein and a second entry indicates a biomarker indicating a diagnostic biomarker (e.g., white blood cell count and/or the like), the one or more sub-entries can represent increases or decreases measured for each biomarker, respectively at the points in time.

At operation 204, the analytics server can determine a set of attributes associated with each patient for each treatment profile. For example, the analytics server can determine a set of attributes, where each attribute is associated with at least one biomarker (e.g., a single biomarker, a combination of biomarkers, and/or the like) that represent the state of the patient. In examples, the analytics server can determine a set of attributes, where each attribute is associated with a combination of biomarkers. In one example, where a patient is expected to have AML, the analytics server can determine a set of attributes for each patient, where the set of attributes represent a plurality of biomarkers (e.g., 300-350 biomarkers) for the patient. In examples, the biomarkers for a given attribute may or may not overlap (e.g., share one or more common biomarkers) with the other biomarkers used to represent the state of the patient.

In some embodiments, the analytics server can determine a set of attributes associated with each patient based on processing of the samples of the patient and determining values representing the one or more biomarkers indicative of the state of the health of the patient. For example, the samples associated with each treatment profile can be prepared for analysis (e.g., at a laboratory system that is the same as, or similar to, the laboratory system 112 of FIG. 1). Analysis can include isolating specific cell types, extracting RNA/DNA to identify one or more genes (e.g., CD70 which encodes the CD70 protein), determining one or more measurements indicative of one or more biomarkers (e.g., measurements quantifying proteins that, when expressed, indicate the potential for one or more diseases to be present), and/or the like. For example, when analyzing samples to identify CD70 protein expression, the samples can be processed based on applying immunohistochemistry or flow cytometry techniques to the samples (e.g., tissue and/or cell samples) using antibodies specific to CD70. For genetic analysis of the samples, RNA or DNA sequencing can be used to identify CD70 gene expression levels. In examples, other techniques including single-cell RNA sequencing can be implemented to pinpoint CD70 expression in specific cell populations. The results generated in response to processing the samples can then be analyzed to determine CD70 expression levels, comparing the expression levels against established thresholds or controls to determine whether a given patient or set of patients expresses CD70 in a manner that is indicative of the presence of one or more cancers. In other examples, the samples can be processed to determine one or more diagnostic biomarkers. For example, the samples can be processed to determine blood glucose levels, cholesterol levels, and/or the like that can be correlated with one or more biomarkers associated with a disease as described herein. In other examples, the samples can be processed to determine one or more cytogenetic abnormalities, biochemical markers (e.g., leukocytosis) and/or the like. The analytics server can then determine values and/or the like for the entries of the treatment profile for each patient based on the results of the analysis of the sample(s) collected from each patient.

At operation 206, the analytics server can, in response to determining that a first biomarker is present, determine correlations between a subset of the attributes for each patient of the set of patients. For example, the analytics server can determine that a first biomarker is present for the set of patients, where the first biomarker is indicative of a disease (e.g., AML and/or the like). In some embodiments, the analytics server can determine that the first biomarker is present based on the analytics server comparing a value associated with the first biomarker represented by a given treatment profile to a measurement indicating the presence or expression level of a gene (e.g., CD70) that encodes a protein, an expression level of the protein, or any other quantifiable biomarker. The analytics server can then compare the value associated with the first biomarker one or more predetermined threshold measurements or threshold ranges of measurements to determine whether the predetermined threshold measurement or threshold range of measurements are satisfied. In an example, where the expression level of a protein satisfies an expression level threshold, the analytics server can determine that the biomarker (e.g., the protein) is present in sufficient quantity to indicate that a disease is present; and where the expression level for the biomarker does not satisfy the expression level threshold, the analytics server can determine that the biomarker is not present in sufficient quantity. In some embodiments, the analytics server can then determine correlations between a subset of the attributes for each patient of the set of patients based on the determination that the biomarker is present and satisfies a predetermined expression level.

In some embodiments, the analytics server can determine the correlations between the subsets of the attributes for each patient of the set of patients based on clusters and/or sub-clusters of the treatment profiles associated with the set of patients. For example, the analytics server can cluster the treatment profiles of the set of patients into clusters and/or sub-clusters. The analytics server can cluster the treatment profiles based on similarities between one or more by assigning the treatment profiles with similar attributes to corresponding clusters and/or sub-clusters. In one example, the analytics server assign treatment profiles that are associated with (e.g., have) one or more first attributes that satisfy a primary correlation threshold to a first cluster. In this example, the one or more first attributes can satisfy the primary correlation threshold when at least one biomarker associated with the one or more first attributes satisfies a threshold measurement or threshold range of measurements. Satisfaction of the threshold measurement or threshold range of measurements can indicate that the corresponding treatment profiles are associated with patients that have a disease. With continued reference to this example, the analytics server can assign each treatment profile of the first cluster to one or more sub-clusters. For example, the analytics server can assign each treatment profile of the first cluster to one or more sub-clusters based on similarities between one or more different attributes (e.g., second biomarkers) represented by the treatment profiles. In an example, the one or more second biomarkers can include a single biomarker. In this example, the analytics server can assign the treatment profiles of the first cluster when the single biomarker represented by the attributes of each treatment profile satisfies a second threshold measurement or threshold range of measurements. Satisfaction of the second threshold measurement or threshold range of measurements can indicate that each treatment profile of the one or more sub-clusters are associated with aspects (e.g., biomarker measurements) that are similar to the other treatment profiles of the respective one or more sub-clusters. In another example, the one or more second biomarkers can include a set of (e.g., a combination of) biomarkers that are each associated with a respective threshold measurements or threshold ranges of measurements, and the analytics server can assign the treatment profiles of the first cluster that express or satisfy the respective thresholds or threshold ranges of measurements to the corresponding sub-cluster(s). In this example, satisfaction of the respective thresholds or threshold ranges of measurements can indicate that each treatment profile of the one or more sub-clusters are similar to the other treatment profiles of the respective one or more sub-clusters. In this way, the analytics server can cluster the treatment profiles into sub-clusters where each treatment profile satisfies a threshold or threshold range for a given biomarker or combination of biomarkers. The analytics server can then determine that the one or more subsets of attributes indicated by treatment profiles of one or more sub-clusters are indicative of the disease. As a result, the analytics server can cluster treatment profiles into one or more sub-clusters and determine that biomarkers not predetermined as being associated with the disease are indicative of the disease.

In examples, the analytics server can determine a first set of correlations between a first subset of attributes of a plurality of treatment profiles, and a second set of correlations between a second subset of attributes represented by the plurality of treatment profiles. For example, the analytics server can determine the first set of correlations, where the first set of correlations are predetermined to be indicative of a disease. In this example, the analytics server can determine the first set of correlations based on the analytics server determining that the first subset of attributes for each treatment profile includes biomarkers that satisfy a primary correlation threshold (e.g., a set of thresholds or threshold ranges corresponding to each biomarker of the first subset of biomarkers). In this example, the treatment profiles of the plurality of treatment profiles that satisfy the primary correlation threshold can be predetermined to indicate the disease with a greater likelihood than the treatment profiles that include fewer biomarkers that satisfy similar thresholds. The analytics server can then determine a second set of correlations between the treatment profiles based on the first set of correlations. For example, the analytics server can filter the treatment profiles from the plurality of treatment profiles that do not satisfy the primary correlation threshold. The analytics server can then determine the second set of correlations based on the analytics server determining similarities between a second subset of biomarkers represented by the treatment profiles (e.g., that do satisfy the primary correlation threshold). In this example, the analytics server can determine the second set of correlations based on the analytics server comparing the values of the biomarkers for the treatment profiles to determine one or more clusters, distance metrics, similarities (e.g., cosine similarities), and/or the like. As a result, the analytics server can determine that, in cases where a treatment profile includes biomarkers that satisfy the secondary correlation threshold, the treatment profile is indicative of the disease. In this way, the analytics server can be configured to identify correlations between biomarkers that are not predetermined to be associated with the disease as being indicative of the disease.

In some embodiments, the analytics server can determine correlations between a subset of the attributes of the set of treatment profiles indicative of the disease over a period of time. For example, the analytics server can determine changes across each attribute of the treatment profiles that occur over the period of time. These changes can include differences represented by changes in values (e.g., quantities of measured biomarkers) across points in time over the period of time (e.g., increases or decreases in gene expression that are indicative of disease progression), differences in rates of change between the values and the points in time (e.g., increases or decreases in rates of change of gene expression that are indicative of disease progression), and/or the like. The analytics server can then determine the correlations between the subsets of attributes based on the changes across each attribute over the period of time. As a result, the analytics server can determine that treatment profiles that are associated with similar changes are indicative of the disease.

In examples, to determine the correlations between the subset of attributes indicative of the disease over the period of time based on latent patterns indicated by the attributes, the analytics server can determine a first attribute that is associated with a first change pattern over the period of time. The first change pattern can represent changes in values (e.g., quantities) for a given biomarker or set of biomarkers over the period of time, changes in rates of change for the given biomarker or set of biomarkers over the period of time, and/or the like. For example, for treatment profiles associated with progression of the disease, the analytics server can determine that the biomarkers of the first attribute differ by one or more predetermined values across points in time, at one or more predetermined rates across the points in time, and/or the like in accordance with the progression of the disease. In this example, the predetermined values and/or the predetermined rates can be determined by the analytics server as indicative of progression of the disease as described herein. In the examples described herein, the first change pattern can be predetermined as being associated with the disease or can be identified by the analytics server as being associated with the disease.

In examples, the analytics server can determine a second attribute that is associated with a second change pattern over the period of time. The second change pattern can represent changes in values for a given biomarker or set of biomarkers over the period of time, changes in rates of change for the given biomarker or set of biomarkers over the period of time, and/or the like, as described with respect to the first change pattern. In this example, the analytics server can determine the second change pattern as indicative of progression of the disease based on a correlation between the second change pattern and the first change pattern. For example, where the first change pattern is associated with changes in values, rates of change, and/or the like of the biomarkers associated with the first attribute, the second change pattern can be associated with changes in values, rates of change, and/or the like of a biomarker and/or a set of biomarkers that are different from the biomarkers representing the first attribute. As a result, the analytics server can determine that, in cases where a treatment profile includes biomarkers that are similar to the second change pattern, the analytics server can determine that the biomarkers of the treatment profile are indicative of the disease.

At operation 208, the analytics server can provide output data associated with the correlations indicating the subset of biomarkers that are indicative of the disease. For example, the analytics server can generate the output data based on the analytics server determining the correlations and corresponding biomarkers that are indicative of the disease as described herein. In this example, the output can include an indication of biomarkers or combinations of biomarkers (and their corresponding thresholds or threshold ranges) that, when satisfied by entries of a treatment profile for a patient, indicate that the patient has the disease. In an example, the analytics server can provide the output data to cause a graphical user interface (GUI) of a computing device (e.g., a client device that is the same as, or similar to, the client device 126 of FIG. 1) to be displayed and indicate the subset of biomarkers that are indicative of the disease. In another example, the analytics server can provide the output data to cause a sample analysis device (e.g., a laboratory system, a sequencing system, and/or a client device) to compare the indication of biomarkers or combinations of biomarkers (and their corresponding thresholds or threshold ranges) such that, when analyzing subsequently-received treatment profiles, the sample analysis device compares the entries of the subsequently-received treatment profiles to the biomarkers and indicates whether the patient has the disease.

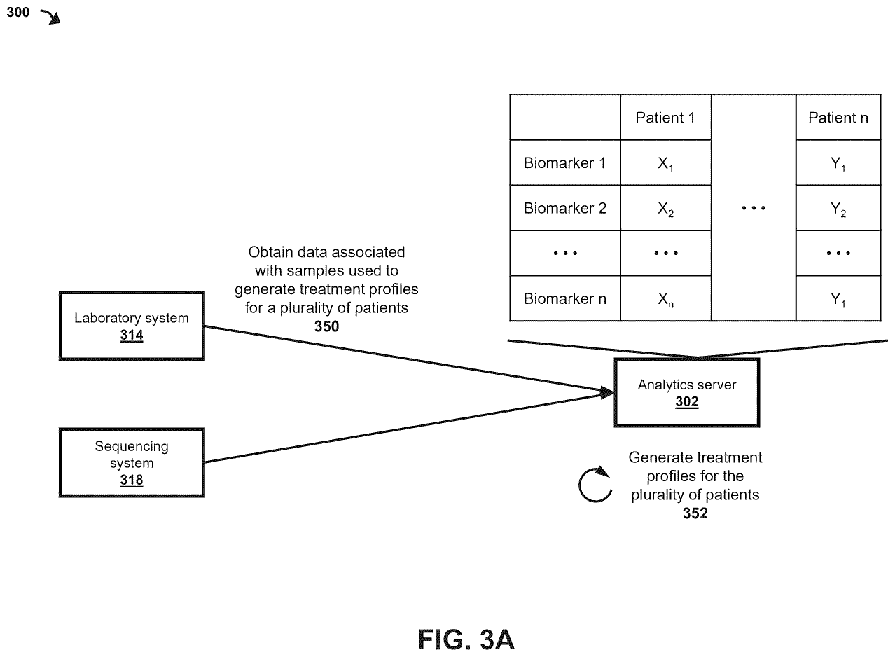

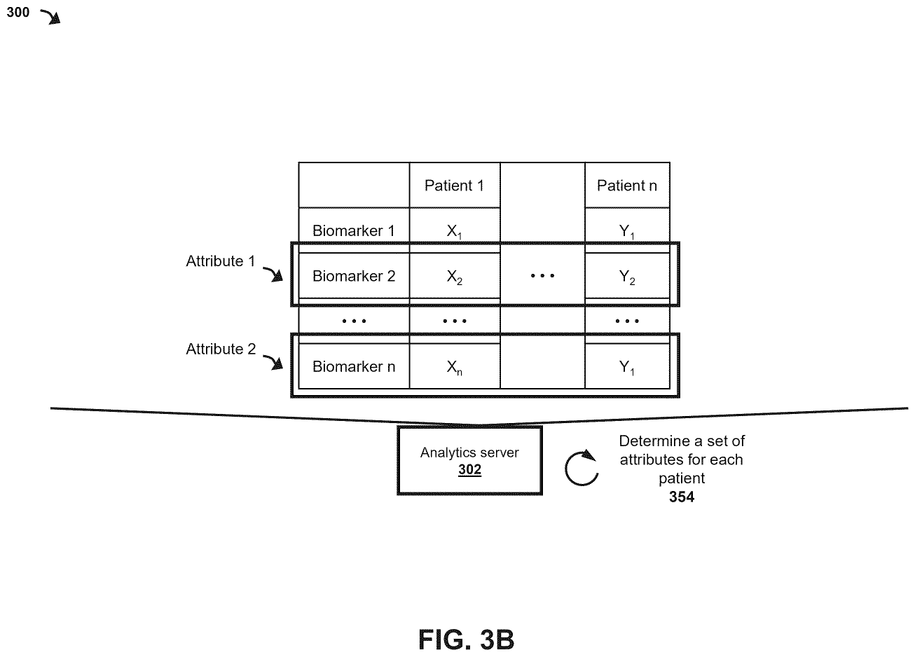

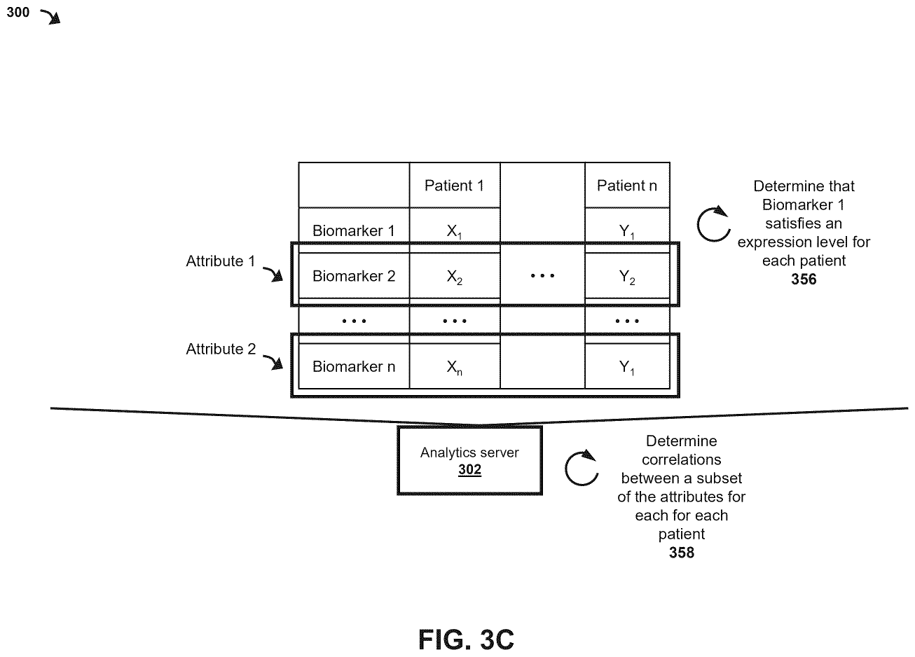

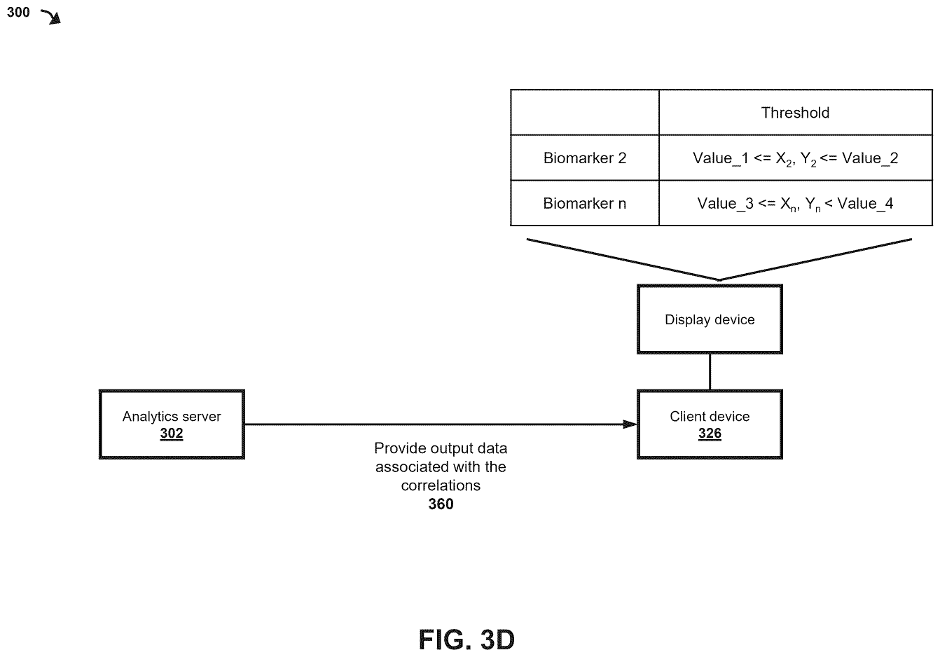

FIG. 3A-3D are a diagram of an example implementation 300 of the method of FIG. 2, in accordance with one or more embodiments described herein. In some embodiments, the operations of the implementation 300 can be implemented by an analytics server 302, a global patient database 310, a laboratory system 314, and a sequencing system 318 that are the same as, or similar to, the analytics server 102, the global patient database 110, the laboratory system 112, and the sequencing system 118 of FIG. 1. Additionally, or alternatively, one or more of the operations of the implementation 300 can involve a data integration engine 304, a global patient database 310 and/or a sequence database 319 that are the same as, or similar to, the data integration engine 104, a global patient database 110 and/or a sequence database 119 of FIG. 1.

At operation 350, the analytics server 302 can obtain data associated with patient samples for a plurality of patients (e.g., Patient 1-Patient n) and/or data associated with the sequenced DNA of the plurality of patients from a laboratory system 314 and/or a sequencing system 318 (each of which can represent data that is gathered as described herein from one or more individuals and/or patient). For example, patients that are or are not suspected of, being treated for, or at risk for one or more diseases such as AML and/or the like can provide patient samples as described herein for analysis by the laboratory system 314 and/or the sequencing system 318. The samples can be analyzed by the laboratory system 314 and/or the sequencing system 318 to determine one or more values for one or more predetermined biomarkers (e.g., Biomarker 1-Biomarker n). For example, biomarkers indicative of the expression of a protein can be represented using a value to indicate the expression level of the protein. In another example, biomarkers such as diagnostic biomarkers can be represented as values (e.g., white blood cell counts) or percents (e.g., in the context of white blood cells, as a percentage of each type of white blood cell (neutrophils, lymphocytes, monocytes, eosinophils, and basophils)). The laboratory system 314 and/or the sequencing system 318 can provide the data associated with the biomarkers to the analytics server 302 to allow the analytics server 302 to generate a plurality of treatment profiles as described herein. In some embodiments, the analytics server 302 can also obtain patient data indicative of one or more observed conditions of the patient provided as input by a clinician to a client device in communication with the analytics server 302. For example, a clinician can provide input to the analytics server 302 indicating that the patient is experiencing one or more symptoms (e.g., fatigue, dizziness, and/or the like) and those symptoms can be represented as values in the treatment profile (not expressly illustrated). In these examples, the symptoms can be correlated in a manner similar to the biomarkers as described throughout the present disclosure.

At operation 352, the analytics server 302 can generate treatment profiles for the plurality of patients. For example, the analytics server 302 can generate the treatment profiles for the patient based on the corresponding biomarkers of the plurality of patients. In examples, the analytics server 302 can normalize the values associated with the biomarkers of the plurality of patients. In some embodiments, the analytics server 302 can generate a plurality of limited treatment profiles for each corresponding treatment profile, as described herein.

At operation 354, the analytics server 302 can determine a set of attributes for each patient. For example, the analytics server 302 can determine a set of attributes for each patient, where each attribute represents a respective biomarker (e.g., Attribute 1: Biomarker 2, Attribute 2: Biomarker n). In this example, the attributes can represent each biomarker at a point in time represented by the treatment profile of the respective patients. Additionally, or alternatively, the attributes can represent each biomarker and/or changes to each biomarker over a period of time (e.g., a period and/or sub-period during which the patient is evaluated and/or treated for the disease). In some embodiments, the attributes can represent combinations of biomarkers. For example, the attributes can represent combinations of biomarkers that, when satisfying one or more correlation thresholds as described herein, can indicate the presence of the disease of the patients.

At operation 356, the analytics server 302 can determine that one or more biomarkers satisfy an expression level for each patient represented by a set of treatment profiles. For example, the analytics server 302 can determine that one or more biomarkers (e.g., Biomarker 1) are represented with values indicative of a disease. In an example, in the context of AML, the analytics server 302 can determine that a protein such as CD70 is expressed (e.g., as a concentration such as ng/mL, a number of molecules per cell, and/or the like) and the value indicating the expression satisfies one or more thresholds indicative of AML.

At operation 358, the analytics server 302 can determine correlations between a subset of the attributes for each patient. For example, the analytics server 302 can determine correlations between a subset of the attributes for each patient based on the analytics server 302 determining that each patient is associated with a biomarker that is indicative of the disease. In an example, the correlations can include scenarios where one or more attributes are associated with a first threshold or threshold range of values, and/or one or more different attributes are associated with a second threshold or threshold range of values. [EXAMPLE].

At operation 360, the analytics server 302 can provide output data associated with the correlations between the attributes of the treatment profiles identified as representing states of patients with the disease. For example, the analytics server 302 can provide the output data to a client device 326 to cause the client device 326 to generate, via a connected display device, a GUI. The GUI can indicate the correlation. More specifically, the GUI can indicate the biomarkers that, when satisfying one or more thresholds or threshold ranges (illustrated as Value_1<=X₂, Y₂<=Value_2 for Biomarker 1, and Value_3<=Xₙ, Yₙ<Value_4 for Biomarker 2), indicate that patients with similar biomarkers (e.g., that satisfy similar thresholds or threshold ranges) have the disease. In other examples, the analytics server 302 can provide the output data to cause a sample analysis device (e.g., the laboratory system 314, a sequencing system 318, and/or a client device 326) to compare the indication of biomarkers or combinations of biomarkers (and their corresponding thresholds or threshold ranges) such that, when analyzing subsequently-received treatment profiles, the sample analysis device compares the entries of the subsequently-received treatment profiles to the biomarkers and indicates whether the patient has the disease.

The various illustrative logical blocks, modules, circuits, and algorithm steps described in connection with the embodiments disclosed herein can be implemented as electronic hardware, computer software, or combinations of both. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans can implement the described functionality in varying ways for each particular application, but such implementation decisions should not be interpreted as causing a departure from the scope of this disclosure or the claims.

Embodiments implemented in computer software can be implemented in software, firmware, middleware, microcode, hardware description languages, or any combination thereof. A code segment or machine-executable instructions can represent a procedure, a function, a subprogram, a program, a routine, a subroutine, a module, a software package, a class, or any combination of instructions, data structures, or program statements. A code segment can be coupled to another code segment or a hardware circuit by passing and/or receiving information, data, arguments, parameters, or memory contents. Information, arguments, parameters, data, etc., can be passed, forwarded, or transmitted via any suitable means, including memory sharing, message passing, token passing, network transmission, etc.

The actual software code or specialized control hardware used to implement these systems and methods is not limiting of the claimed features or this disclosure. Thus, the operation and behavior of the systems and methods were described without reference to the specific software code being understood that software and control hardware can be designed to implement the systems and methods based on the description herein.

When implemented in software, the functions can be stored as one or more instructions or code on a non-transitory computer-readable or processor-readable storage medium. The steps of a method or algorithm disclosed herein can be embodied in a processor-executable software module, which can reside on a computer-readable or processor-readable storage medium. A non-transitory computer-readable or processor-readable media includes both computer storage media and tangible storage media that facilitate the transfer of a computer program from one place to another. A non-transitory processor-readable storage media can be any available media that can be accessed by a computer. By way of example, and not limitation, such non-transitory processor-readable media can comprise RAM, ROM, EEPROM, CD-ROM or other optical disk storage, magnetic disk storage or other magnetic storage devices, or any other tangible storage medium that can be used to store desired program code in the form of instructions or data structures and that can be accessed by a computer or processor. Disk and disc, as used herein, include compact disc (CD), laser disc, optical disc, digital versatile disc (DVD), floppy disk, and Blu-ray disc where disks usually reproduce data magnetically, while discs reproduce data optically with lasers. Combinations of the above should also be included within the scope of computer-readable media. Additionally, the operations of a method or algorithm can reside as one or any combination or set of codes and/or instructions on a non-transitory processor-readable medium and/or computer-readable medium, which can be incorporated into a computer program product.

The preceding description of the disclosed embodiments is provided to enable any person skilled in the art to make or use the embodiments described herein and variations thereof. Various modifications to these embodiments will be readily apparent to those skilled in the art, and the principles defined herein can be applied to other embodiments without departing from the spirit or scope of the subject matter disclosed herein. Thus, the present disclosure is not intended to be limited to the embodiments shown herein but is to be accorded the widest scope consistent with the following claims and the principles and novel features disclosed herein.

While various aspects and embodiments have been disclosed, other aspects and embodiments are contemplated. The various aspects and embodiments disclosed are for purposes of illustration and are not intended to be limiting, with the true scope and spirit being indicated by the following claims.

What is claimed is:

1. A system for processing profiles included in a protected dataset maintained in a secured network location and configured to prevent re-identification of individuals represented by the profiles to determine correlations between indicators representing latent patterns that are indicative of a condition being present in individuals, the system comprising:
one or more processors configured to:
obtain data associated with a set of individual profiles corresponding to a set of individuals, where each individual profile of the set of individual profiles comprises a plurality of entries representing a state of an individual;
determine a set of attributes associated with each individual corresponding to each individual profile, where each attribute is associated with at least one indicator representing the state of the individual;
in response to determining that a first indicator is present for the set of individuals based on the first indicator satisfying an expression level for each individual, determine correlations between a subset of the attributes for each individual of the set of individuals that are being treated for a condition, the correlations indicating a subset of indicators different from the first indicator that are indicative of the condition; and
provide output data associated with the correlations indicating the subset of indicators, the output data configured to cause a graphical user interface (GUI) to be displayed that indicates subset of indicators that are indicative of the condition.

2. The system of claim 1, wherein the one or more processors configured to determine the correlations between the subset of the attributes are configured to:
cluster the individual profiles to form a plurality of clusters, where each individual profile is assigned to a cluster of the plurality of clusters based on similarities between attributes of the subset of attributes, and
determine the correlations between the subset of the attributes based on the plurality of clusters.

3. The system of claim 2, wherein the one or more processors configured to determine the correlations between the subset of the attributes based on the plurality of clusters are configured to:
perform a cluster analysis based on the plurality of clusters to identify attributes that are indicative of presence of the condition, and
determine the subset of indicators that correspond to the attributes that are indicative of the presence of the condition.

4. The system of claim 1, wherein the one or more processors configured to determine the correlations between the subset of the attributes are configured to:
determine a first set of correlations indicating a first subset of indicators, where each indicator of the first subset of indicators satisfies a primary correlation threshold indicative of the condition, and
wherein the one or more processors are further configured to:
determine a second set of correlations indicating a second subset of indicators, where each indicator of the second subset of indicators satisfies a secondary correlation threshold indicative of the condition, and where each indicator of the second subset of indicators is not indicated by the first subset of indicators.

5. The system of claim 1, wherein the one or more processors configured to obtain the data associated with a set of individual profiles corresponding to a set of individuals are configured to:
obtain the data associated with a set of individual profiles, where each individual profile comprises a plurality of entries having one or more sub-entries that represent the state of the individual over a period of time, and
wherein the one or more processors configured to determine the correlations between the subset of the attributes are configured to:
determine correlations between the subset of the attributes for individuals over the period of time, the correlations indicating changes in the subset of indicators that are indicative of the condition.

6. The system of claim 5, wherein the one or more processors configured to determine the correlations between the subset of the attributes for individuals over the period of time are configured to:
determine a first attribute that is associated with a change pattern over the period of time, the change pattern representing changes in quantity of an indicator represented by samples gathered from individuals over the period of time.

7. The system of claim 5, wherein the one or more processors configured to determine the correlations between the subset of the attributes for individuals over the period of time are configured to:
determine a first attribute that is associated with a first change pattern over the period of time, the first change pattern representing first changes in quantity of a first indicator represented by samples gathered from individuals over the period of time;
determine a second attribute that is associated with a second change pattern over the period of time, the second change pattern representing first changes in quantity of a second indicator represented by samples gathered from individuals over the period of time; and
determine a correlation between the first attribute and the second attribute based on a comparison of the first changes in quantity and the second changes in quantity over time.

8. The system of claim 7, wherein the first attribute is further associated with a correlation that satisfies a primary correlation threshold indicative of the condition, and
wherein the second attribute is further associated with correlation that satisfies the primary correlation threshold or a secondary correlation threshold indicative of the condition.

9. A method for processing profiles included in a protected dataset maintained in a secured network location and configured to prevent re-identification of individuals represented by the profiles to determine correlations between indicators representing latent patterns that are indicative of a condition being present in individuals, the method comprising:
obtaining, by one or more processors, data associated with a set of individual profiles corresponding to a set of individuals, where each individual profile of the set of individual profiles comprises a plurality of entries representing a state of an individual;
determining, by the one or more processors, a set of attributes associated with each individual corresponding to each individual profile, where each attribute is associated with at least one indicator representing the state of the individual;
in response to determining that a first indicator is present for the set of individuals based on the first indicator satisfying an expression level for each individual, determining, by the one or more processors, correlations between a subset of the attributes for each individual of the set of individuals that are being treated for a condition, the correlations indicating a subset of indicators different from the first indicator that are indicative of the condition; and
providing, by the one or more processors, output data associated with the correlations indicating the subset of indicators, the output data configured to cause a graphical user interface (GUI) to be displayed that indicates subset of indicators that are indicative of the condition.

10. The method of claim 9, wherein determining the correlations between the subset of the attributes comprises:
clustering, by the one or more processors, the individual profiles to form a plurality of clusters, where each individual profile is assigned to a cluster of the plurality of clusters based on similarities between attributes of the subset of attributes, and
determining, by the one or more processors, the correlations between the subset of the attributes based on the plurality of clusters.

11. The method of claim 10, wherein determining the correlations between the subset of the attributes based on the plurality of clusters comprises:
performing, by the one or more processors, a cluster analysis based on the plurality of clusters to identify attributes that are indicative of presence of the condition, and
determine, by the one or more processors, the subset of indicators that correspond to the attributes that are indicative of the presence of the condition.

12. The method of claim 9, wherein determining the correlations between the subset of the attributes includes:
determining, by the one or more processors, a first set of correlations indicating a first subset of indicators, where each indicator of the first subset of indicators satisfies a primary correlation threshold indicative of the condition, and
the method further comprising:
determining, by the one or more processors, a second set of correlations indicating a second subset of indicators, where each indicator of the second subset of indicators satisfies a secondary correlation threshold indicative of the condition, and where each indicator of the second subset of indicators is not indicated by the first subset of indicators.

13. The method of claim 9, wherein obtaining the data associated with a set of individual profiles corresponding to a set of individuals comprises:
obtaining, by the one or more processors, the data associated with a set of individual profiles, where each individual profile comprises a plurality of entries having one or more sub-entries that represent the state of the individual over a period of time, and
wherein determining the correlations between the subset of the attributes comprises:
determining, by the one or more processors, correlations between the subset of the attributes for individuals over the period of time, the correlations indicating changes in the subset of indicators that are indicative of the condition.

14. The method of claim 13, wherein determining the correlations between the subset of the attributes for individuals over the period of time comprises:
determining, by the one or more processors, a first attribute that is associated with a change pattern over the period of time, the change pattern representing changes in quantity of an indicator represented by samples gathered from individuals over the period of time.

15. The method of claim 13, wherein determining the correlations between the subset of the attributes for individuals over the period of time comprises:
determining, by the one or more processors, a first attribute that is associated with a first change pattern over the period of time, the first change pattern representing first changes in quantity of a first indicator represented by samples gathered from individuals over the period of time;
determining, by the one or more processors, a second attribute that is associated with a second change pattern over the period of time, the second change pattern representing first changes in quantity of a second indicator represented by samples gathered from individuals over the period of time; and
determining, by the one or more processors, a correlation between the first attribute and the second attribute based on a comparison of the first changes in quantity and the second changes in quantity over time.

16. The method of claim 15, wherein the first attribute is further associated with a correlation that satisfies a primary correlation threshold indicative of the condition, and
wherein the second attribute is further associated with correlation that satisfies the primary correlation threshold or a secondary correlation threshold indicative of the condition.

17. A non-transitory computer-readable medium storing instructions thereon that, when executed by one or more processors, cause the one or more processors to:
obtain data associated with a set of individual profiles corresponding to a set of individuals, where each individual profile of the set of individual profiles comprises a plurality of entries representing a state of an individual;
determine a set of attributes associated with each individual corresponding to each individual profile, where each attribute is associated with at least one indicator representing the state of the individual;
in response to determining that a first indicator is present for the set of individuals based on the first indicator satisfying an expression level for each individual, determine correlations between a subset of the attributes for each individual of the set of individuals that are being treated for a condition, the correlations indicating a subset of indicators different from the first indicator that are indicative of the condition; and
provide output data associated with the correlations indicating the subset of indicators, the output data configured to cause a graphical user interface (GUI) to be displayed that indicates subset of indicators that are indicative of the condition.

18. The non-transitory computer-readable medium of claim 17, wherein the instructions that cause the one or more processors configured to determine the correlations between the subset of the attributes cause the one or more processors to:
cluster the individual profiles to form a plurality of clusters, where each individual profile is assigned to a cluster of the plurality of clusters based on similarities between attributes of the subset of attributes, and
determine the correlations between the subset of the attributes based on the plurality of clusters.

19. The non-transitory computer-readable medium of claim 18, wherein the instructions that cause the one or more processors configured to determine the correlations between the subset of the attributes based on the plurality of clusters cause the one or more processors to:
perform a cluster analysis based on the plurality of clusters to identify attributes that are indicative of presence of the condition, and
determine the subset of indicators that correspond to the attributes that are indicative of the presence of the condition.

20. The non-transitory computer-readable medium of claim 17, wherein the instructions that cause the one or more processors configured to determine the correlations between the subset of the attributes cause the one or more processors to:
determine a first set of correlations indicating a first subset of indicators, where each indicator of the first subset of indicators satisfies a primary correlation threshold indicative of the condition, and
wherein the instructions further cause the one or more processors to:
determine a second set of correlations indicating a second subset of indicators, where each indicator of the second subset of indicators satisfies a secondary correlation threshold indicative of the condition, and where each indicator of the second subset of indicators is not indicated by the first subset of indicators.
